(19) United States
(12) Patent Application Publication
(10) Pub. No.: US 2026/0114783 A1
(43) Pub. Date: Apr. 30, 2026

(54) SYSTEMS AND METHODS FOR MEDICAL DATA COLLECTION, STORAGE, REGULATION, ANALYSIS, AND PROCESSING

(71) Applicant: Spaulding Medical, LLC, West Bend, WI (US)

(72) Inventors: Nicholas Alexander Fetcko, West Bend, WI (US); Randol R. Spaulding, West Bend, WI (US); Mark Geisler, Franklin, WI (US)

(73) Assignee: Spaulding Medical, LLC, West Bend, WI (US)

(21) Appl. No.: 19/229,859

(22) Filed: Jun. 5, 2025

Related U.S. Application Data
(60) Provisional application No. 63/656,473, filed on Jun. 5, 2024.

(51) Int. Cl. A61B 5/346; A61B 5/00; A61B 5/339; A61B 90/96; A61B 90/98; G16H 10/60; G16H 50/70

(52) U.S. Cl. CPC A61B 5/346; A61B 5/0006; A61B 5/339; A61B 5/7267; A61B 5/749; A61B 90/96; A61B 90/98; G16H 10/60; G16H 50/70

(57) ABSTRACT

This disclosure is directed to systems and methods for data collection, storage, analysis, and processing, and in particular, collection, storage, regulation, analysis, and processing of sensitive data such as medical data. A data collection device may collect Electrocardiogram (ECG) data and other medical data, and the ECG data and other medical data may be stored remotely for access by authorized devices and entities. Additional data from other data sources can also be stored remotely. The ECG data, other medical data, and additional data can be analyzed and processed to diagnose a patient.

### CROSS-REFERENCE TO RELATED APPLICATION(S)

This application claims the benefit of and priority to U.S. Provisional Application No. 63/656,473, filed on Jun. 5, 2024, titled SYSTEMS AND METHODS FOR MEDICAL DATA COLLECTION, STORAGE, REGULATION, ANALYSIS, AND PROCESSING, the disclosure of which is hereby incorporated by reference in its entirety.

### TECHNICAL FIELD

In general terms, this disclosure relates to data collection, storage, regulation, analysis, and processing.

### BACKGROUND

Electrocardiogram (ECG) systems may collect data for a patient, including the patient's heart rate, heart rhythm, and heart electrical activity. Some ECG systems may also monitor respiratory rate, oxygen saturation, and/or the like. ECG systems are typically large, heavy, and otherwise difficult to transport and may therefore be stationary or confined to an area (e.g., a hospital wing). Thus, ECG systems may not be easily transported for data collection or review where the collection or review is needed and may not be easily used outside of medical facilities, such as at a patient's home.

ECG systems may also collect ECG data in different formats and/or structures, and may not provide the data for access remotely, such as via a network. Therefore, entities authorized to access the data may be unable to access all data available. Additionally, using data from multiple ECG systems may be cumbersome or even impossible. Without access to all data associated with a patient, accurately diagnosing patients can be more difficult and may take longer than necessary.

Additionally, data, such as medical data, can be collected by many sources (e.g., ECG machine, telemetry monitor, scale, blood pressure monitor, continuous glucose monitoring systems, databases, servers, storage devices, patient devices, medical devices, other entity devices, etc.). Specialized devices can collect information at medical facilities, at the patient's home, and in ambulances. The collected data may be collected in one or more formats and/or or structures depending on the device used for collection, and the collected data may not be shared or otherwise accessible between different medical entities and even between separate locations associated with a single medical entity. Different data formats, different data structures, and/or the inaccessibility of collected medical data can lead to redundant data collection, the inability to effectively use the data (e.g., to accurately diagnose patients using all information available), and other issues.

### SUMMARY

The present disclosure relates to data collection, storage, analysis, and processing, and in particular, collection, storage, regulation, analysis, and processing of sensitive data such as medical data. In a non-limiting example, a data collection device and/or additional data sources collect data, and the collected data is stored in a remote location, such as stored on remote servers also known as cloud storage. Authorized devices and entities may view the data, analyze the data, and/or process the data, for example to accurately diagnose patients.

### BRIEF DESCRIPTION OF THE DRAWINGS

The following drawings are illustrative of particular examples of the present disclosure and therefore do not limit the scope of the present disclosure. The drawings are not necessarily to scale and are intended for use in conjunction with the explanations in the following detailed description. Examples of the present disclosure will hereinafter be described in conjunction with the appended drawings, wherein like numerals denote like elements.

FIG. 1 is a block diagram of an operating environment for data collection, storage, analysis, and processing.

FIG. 2 is an illustration of an example data collection device.

FIG. 3 is a login interface for logging in to the data collection device.

FIG. 4 is a patient data interface for identifying patient information.

FIG. 5 is a workflow interface for managing a workflow and uploading data.

FIG. 6 is a patient queue interface for managing patient data collection.

FIG. 7 is a multi-lead medical data interface for viewing and analyzing patient data.

FIG. 8 is a single lead medical data interface for viewing and analyzing patient data.

FIG. 9 is a single-lead medical data interface for inputting interpretations of patient data.

FIG. 10 is an authentication interface for authenticating a user.

FIG. 11 is an interface for viewing and evaluating patient data.

FIG. 12 is a block diagram of a communications flow between systems for data collection, storage, analysis, and processing.

FIG. 13 is a block diagram of a centralized repository for data storage, analysis, and processing.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

Systems and methods are described herein for collecting data from multiple sources, storing the data in a remotely accessible location (e.g., in a data lake), and analyzing the data for diagnostic applications. Data collection devices can be used to collect, store, and analyze the data. In certain embodiments, the methods and systems are directed to sensitive data such as medical data, including Electrocardiogram (ECG) data, patient monitoring data (e.g., telemetry data), ambulatory and acute care data, health records, patient registries, administrative data, clinical trial data, health surveys, and the like. The methods and systems can enable data to be gathered and shared by anyone, anytime, anywhere, while providing data protections for individuals to secure their data.

Data can be collected by different specialists with varying areas of specialty (e.g., a clinical cardiologist, a heart surgeon, an interventional cardiologist, an electrophysiologist, a heart failure specialist, etc.). Each specialist may use different devices for data collection, and each specialist may collect different data based on the purpose of the data collection. The specialists may collect clinical grade data that may be useful for applications that the specific specialist is not using the data for. Therefore, each specialist may be able to select a purpose or modality the specialist is collecting data for and/or intending to review data for. The specialist may then receive access to data collected by other specialists that may be useful for the modality the specialist selected. Thus, a specialist can have access to more data and may be able to more accurately evaluate a patient.

Data can be collected from multiple sources, including machines at medical facilities (e.g., primary care clinics, hospitals, research facilities), patient's homes or place of residence, and ambulances. The data may be collected by many types of devices, including a Holter monitor, three lead, five lead, twelve lead, and fifteen lead ECG systems, heartrate monitors, etc. Thus, the data may be clinical grade data (e.g., data with a high enough accuracy, completeness, relevance, reliability, timeliness, etc.) and/or lower grade data. Clinical grade data trait requirements may be determined by a medical entity, regulatory agency, and/or the like. Each instance of data collection may be used for a specific purpose, such as a medical entity using a heartrate monitor to check a person's heartrate during a yearly physical. However, the collected data can also be stored to be used for other purposes or modalities. For example, the heartrate data collected for a yearly physical can be used for other analysis processes, for example to diagnose the individual with heart disease using the heartrate data and other collected data.

The comprehensive collection of data at any location can enable faster and more efficient data collection. For example, a patient can use systems at home during a telehealth appointment with their doctor instead of having the remote appointment being limited to a videoconference with their medical provider. The data may be collected by multiple systems, including by the data collection device described below, and the data may be collected in multiple formats. In the medical data embodiment, the data can be collected by the data collection device described below, existing medical devices (e.g., a scale, a blood pressure monitor, a continuous glucose monitoring system), general systems (e.g., a computer), and the like.

The collected data can be sent to a centralized repository, such as a data lake, designed to store the data. The data may be stored in many formats, and the data can be structured, semi structured, and/or unstructured. The data can be catalogued, indexed, or otherwise organized for analysis without data movement. Additionally, the data can be organized as needed, such as when a specific set of data is to be used for analysis. As described above, the collected data can be clinical grade data and/or lower grade data. The data can be stored according to the grade of the data so the data can be effectively used for different purposes.

The centralized repository may consist of remote servers (e.g., a cloud service), storage devices, and the like. The central repository may be a collection of databases hosted on multiple computers, on cloud storage, a combination of the two, and/or the like.

The data can be secured so that individuals' data can only be accessed by authorized entities. Individuals can provide and revoke authorization to allow or deny an entity access to data, such as an individual's health record that can include any of the stored data associated with the individual. An entity can be associated with data collection and may automatically have access to the associated data. For example, ECG data collected at a hospital will be able to be accessed by the hospital. The hospital's access may be restricted however (e.g., the individual must approve sharing data with other entities).

A separate entity and/or a medical entity may control the centralized repository. The entity that controls the centralized repository and approved third party entities can access, analyze, and process the data stored in the centralized repository. Analysis and processing can be done remotely, enabling remote patient monitoring and the use of data collected from anywhere. In the medical data embodiment, the data can be analyzed to gain insights into patient health and care and to generate preventative care, diagnostic care, and treatment recommendations. The data of multiple individuals can be used in the analysis process. For example, the heart rate data of multiple individuals can be compared to determine whether an individual is exhibiting a normal heart rate.

Algorithms, such as machine learning algorithms, and the like can be used to perform data analysis and processing. Additionally, the data can be used to train and otherwise improve the machine learning techniques and algorithms. In embodiments, the data can be depersonalized when used to train the machine learning techniques and algorithms to protect the individuals' sensitive information while allowing the machine learning techniques and algorithms to be improved. The data can also be depersonalized when data associated with multiple individuals is used to gain insights about a specific individual.

The data may be used to perform real time alerting without the need for specialist review before the alert is sent. For example, the data may be analyzed as it is collected to determine if the data indicates that the patient has any health concerns. The alert may be sent to a specialist for review to determine if the flagged health concern is present in the patient.

The data collection device can perform the data collection, storage, and analysis methods described above. The system can collect multiple types of data. For example, the system can collect ECG data, Holter and event monitoring data, vital sign data (e.g., heart rate, blood pressure, temperature), telemetry monitoring data, cardiac stress testing data.

The data collection device can be portable, such as a tablet or a smartphone. Thus, the system can be used in many locations (e.g., primary care clinics, hospitals, research facilities, patient's homes or place of residence, and ambulances) and transported between the locations. Multiple collection devices can be used and interchanged to collect data associated with single individual and store the data, and the stored data is identifiable as associated with the individual even when multiple devices are used for collection. The system may store the data locally and upload the data to the centralized repository when the system is connected to a network that allows the system to transfer the data.

The data collection device can include connections for several types of sensors, such as connections for leads used to collect ECG data. The data collection device can also include standardized connections (e.g., a Universal Serial Bus Type-C (USB-C) connection) that are programmed to be used to collect data when connected to different sensors.

The data collection device can display the collected data, including previously collected data and/or live data collected by the data collection device and/or other systems. The system can also display the analysis performed by the machine learning techniques, algorithms, and other analysis methods described above. Users of the data collection device and/or other systems (e.g., medical professionals) can review the displayed data and data analysis and manipulate the data to gain insights and generate recommendations.

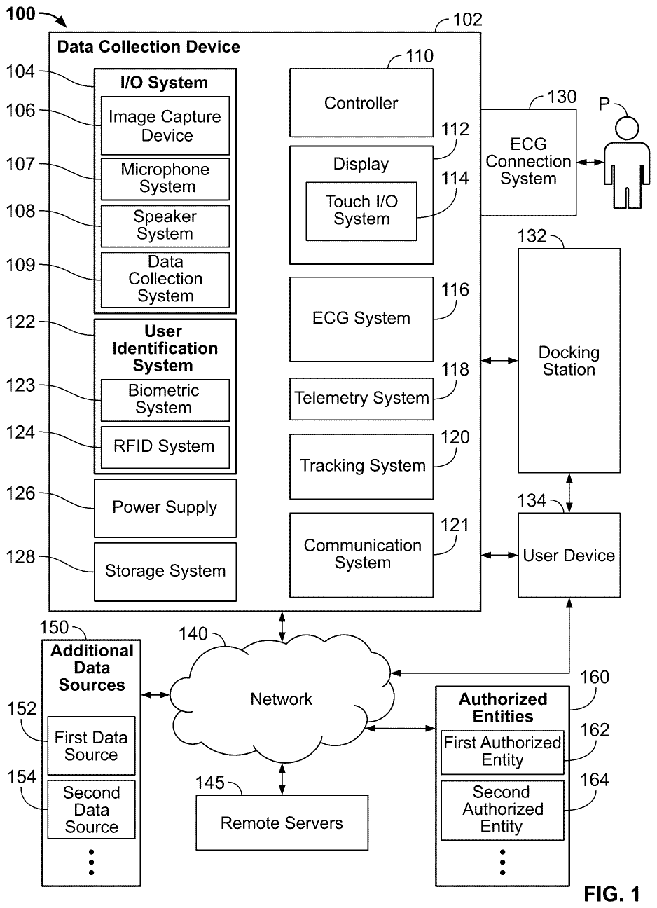

FIG. 1 is a block diagram of an operating environment 100 for data collection, storage, analysis, and processing. The operating environment 100 includes a data collection device 102, a docking station 132, a patient P, a user device 134, a network 140, and remote servers 145. The operating environment 100 also includes additional data sources 150, including a first data source 152, and a second data source 154. There may be more or fewer additional data sources 150 in other examples. The additional data sources 150 may be additional data collection device 102 and/or other data sources. The operating environment 100 also includes authorized entities 160, including a first authorized entity 162 and a second authorized entity 164. There may be more or fewer authorized entities 160 additional examples. The data collection device 102 includes an Input/Output (I/O) system 104, a controller 110, a display 112, an Electrocardiogram (ECG) system 116, a telemetry system 118, a tracking system 120, a communication system 121, a user identification system 122, a power supply 126, and a storage system 128. The controller 110 can control the operation of the data collection device 102 and the data collection device 102 components.

The I/O system 104 can control the reception of inputs to the data collection device 102 and the transmission of outputs from the data collection device 102. The I/O system 104 includes an image capture device 106, a microphone system 107, a speaker system 108, and a data collection system 109. The image capture device 106 can capture images, including images of machine-readable codes (e.g., a barcode, a Quick Response (QR) code). The data collection device 102, via the controller 110 for example, may identify patient information (e.g., information associated with the patient P) using an image of a machine-readable code. The machine-readable code may include a pointer to stored patient information, a link to a web address that includes the patient information, and/or the like.

The microphone system 107 may collect audio data, and the data collection device 102 may determine to adjust operations based on the audio data. For example, a user of the data collection device 102 may control the data collection device 102 using voice commands collected by the microphone system 107. A voice command can be a command to collect medical data, a command to stop collecting medical data, a command to display data, etc. The speaker system 108 may output audio. For example, the data collection device 102 may output audio associated with collected and/or displayed medical data, output an alert when medical data indicates a health concern, of the patient P for example, and/or the like.

The data collection system 109 may control the reception of data, by the ECG system 116 via the ECG connection system 130, by the tracking system 120, by the telemetry system 118, and/or the like. The data collection system 109 may identify the connection and the associated data type, and route collected data to the correct system. For example, the data collection system 109 may identify that the ECG connection system 130 is collecting data and ensure that the ECG system 116 is receiving the data. In other examples, the routing of collected data is based on the connection used (e.g., a connection for the ECG connection system 130, a connection for telemetry devices, etc.).

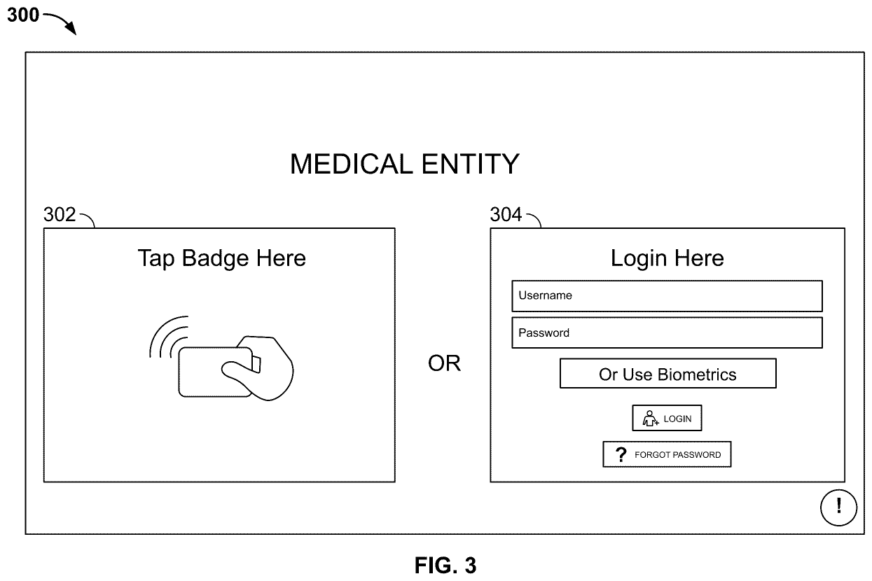

The display 112 may display interfaces, such as Graphical User Interface (GUI) views, and the interfaces may include user login elements, medical data elements, data analysis and/or processing elements, user authentication elements, and/or the like. Example interfaces are described herein with respect to FIGS. 3-11. The display 112 includes a touch I/O system 114 for a user of the data collection device 102 to interact with the displayed interface. The I/O system 104 may control the operation of the touch I/O system 114. In other examples, a user may interact with the displayed interface with other I/O systems, such as a mouse, a keyboard, and/or the like.

The data collection device 102 may receive medical data of the patient P, such as via an ECG connection system 130 sending data from the patient P to the ECG system 116. The data collection device 102 may receive ECG data associated with the patient P using the ECG connection system 130, including heart rate, heart rhythm, and/or heat electrical activity (e.g., Holter monitoring) for example. Thus, the ECG system 116 may be a Holter monitor, an event monitor, a real-time monitor, a resting monitor, an exercise or stress test monitor, and/or the like. The ECG connection system 130 may include a number of leads to connect to the patient P to collect the ECG data (e.g., three leads, five leads, twelve leads, fifteen leads, etc.). The ECG connection system 130 may connect to the data collection device 102 via a standardized connection, such as via a USB-C connection. The ECG system 116 may process the data received via the ECG connection system 130 to display the data, store the data, and/or the like. In an example, the ECG connection system 130 may attach to the patient P and wirelessly transmit data to the data collection device 102.

The data collection device 102 may collect or otherwise receive and/or monitor additional medical data associated with the patient P. For example, the telemetry system 118 may receive respiratory rate, oxygen saturation, and/or other medical data associated with the patient P. Thus, the data collection device 102 may include additional connections to the patient P to collect the additional medical data. The data collection device 102 may therefore monitor any data associated with a patient for different purposes including, for example, cardiac conditions diagnosis (e.g., arrhythmia detection such as atrial fibrillation, atrial flutter, ventricular tachycardia, etc.), detecting Ischemic heart disease, preoperative assessment, postoperative monitoring, critical care settings (e.g., intensive care unit monitoring), hypertension management, syncope evaluation, exercise stress testing, Holter monitoring, ambulatory monitoring, monitoring the effects of medication, screening for cardiac disorders and/or other disorders, athlete pre-participation screening, telemedicine (i.e., performing monitoring remotely, allowing healthcare providers to assess a patient's status from a distance), and/or the like.

The data collection device 102 may be a portable device (e.g., a tablet device, a handheld device, etc.). The tracking system 120 may track the movement of the data collection device 102. For example, the tracking system 120 may include a Global Positioning System (GPS) module, or perform tracking using the communication system 121 (e.g., Wi-Fi positioning, Radio Frequency (RF) Identification (RFID) positioning, cellular positioning). The tracking system 120 can also include an accelerometer to detect the motion of the data collection device 102. For example, the data collection device 102 may be required to be stationary during data collection, and the tracking system 120 may use the accelerometer to determine whether the data collection device 102 is stationary during data collection. In another example, the movement of the patient may be monitored using tracking system 120 and/or other components of the data collection device 102. For example, the data collection device 102 may determine if the patient is standing, sitting, or laying down and detect when the patient changes position, such as getting out of bed. The data collection device 102 may detect the position and movement of the patient, such as the person's position in a hospital, the person's position in an ambulance, and/or the like.

Because the data collection device 102 may be portable, the data collection device 102 may connect to a docking station 132. The docking station 132 may supply power to the data collection device 102, charge the power supply 126, provide connections to the patient P for collection of data, and/or the like. In certain examples, the ECG connection system 130 may be a component of the docking station 132.

The communication system 121 may enable communications to local devices (e.g., the user device 134) and remote devices, such as via the network 140. The communication system 121 may include Wi-Fi capabilities, cellular capabilities, RFID capabilities, and/or the like. Thus, the data collection device 102 may send data to the user device 134, the remote servers 145, the additional data sources 150, and the authorized entities 160.

The user identification system 122 may verify the identity of a data collection device 102 user. The user identification system 122 includes a biometric system 123 and a RFID system 124. The biometric system 123 can receive biometric characteristics (e.g., fingerprint recognition, facial recognition, etc.), and the user identification system 122 can identify the user based on the biometric characteristics. The RFID system 124 can receive an RF signal, such as from an employee badge, and the and the user identification system 122 can identify the user based on the RF signal. The user identification system 122 may identify a user logging into the data collection device 102, such as to use the data collection device 102 to collect data. The user identification system 122 may also identify a user authorized to approve collected data (e.g., a doctor). For example, once data is collected, approval from an authorized user may be required. The authorized user may review the collected data to determine the data collection was accurate, sufficient, and/or the like, and provide a biometric characteristic and/or a RF signal to approve the data. The authorized user may also provide the biometric characteristic and/or RF signal to send the data collection operation that was completed to a billing department.

The power supply 126 may supply power to the data collection device 102. The power supply 126 may be a battery to allow the data collection device 102 to be portable, and the power supply 126 may charge when connected to an external power source, such as via the docking station 132.

The storage system 128 may store instructions for the operation of the data collection device 102, user information, patient information, medical data, and/or the like. For example, the storage system 128 may store collected data before the data collection device 102 sends the data to other devices (e.g., the user device 134, the remote servers 145, the additional data sources 150, the authorized entities 160).

The docking station 132 can dock with the data collection device 102 to provide power, allow the data collection device 102 to connect to other device (e.g., to exchange information, display information on external devices, etc.), provide connections to data collection systems (e.g., connect to sensors that collect data), and/or the like. The docking station 132 can also be a stand to position the data collection device 102, such as next to the patient P.

The data collection device 102 may communicate with or otherwise connect to the user device 134 directly, via the docking station 132, via the network 140, and/or the like. The data collection device 102 may cause the user device 134 to store, display, analyze, and/or process the data. The data collection device 102 may regulate the data and only provide data to the user device 134 when the user device 134 is authorized to access the data.

The remote servers 145 may store data, organize data, analyze data, process data, and/or the like. The data collection device 102, the additional data sources 150, and other systems may send data to and receive data from the remote servers 145. The remote servers 145 may be a centralized repository, such as a data lake, designed to store the data. The remote servers 145 may store data in many formats, and the remote servers 145 can organized the data in structured, semi structured, and/or unstructured implementations. The remote servers 145 can catalogue, index, and/or otherwise organize data for analysis, without data movement for example. The remote servers 145 may organize data as needed. For example, received data may be unorganized when received, and the remote servers 145 may organize a set of data when the set of data is to be used for analysis (e.g., by the data collection device 102). The collected data can be clinical grade data (e.g., ECG and telemetry data from the data collection device 102) and/or lower grade data. The remote servers 145 may store data to the grade of the data so the data can be effectively used for different purposes.

The data collection device 102 and/or the remote servers 145 data can secure data so that individuals' data can only be accessed by the authorized entities, such as the authorized entities 160, the data collection device 102, and the user device 134. Individuals can provide and revoke authorization to allow or deny an entity access to data, such as an individual's health record that can include any of the stored data associated with the individual. For example, a user of the data collection device 102 or the user device 134 may define a list of authorized users that can access the data associated with the patient P.

An entity can be associated with data collection and may automatically have access to the associated data. For example, a medical entity associated with the data collection device 102 is authorized to access data collected by the data collection device 102. The medical entity's access may be restricted however (e.g., the patient P must approve sharing data with other entities such as the authorized entities 160).

A separate entity and/or a medical entity may control the remote servers 145. The entity that controls the remote servers 145 and approved third party entities (e.g., the authorized entities 160) can access, analyze, and process the data stored by the remote servers 145. Analysis and processing can be done remotely, enabling remote patient monitoring and the use of data collected from anywhere. The data collection device 102 and/or the remote servers 145 can analyze data to gain insights into patient health and care and to generate preventative care, diagnostic care, and treatment recommendations. The data of multiple individuals can be used in the analysis process. For example, the data collection device 102 can compare the heart rate data of multiple individuals and the data collected associated with the patient P to determine whether the patient P is exhibiting a normal heart rate.

The data collection device 102, the user device 134, the remote servers 145, the additional data sources 150, and/or the authorized entities 160 can use algorithms, such as machine learning algorithms and techniques, to perform data analysis and processing. Additionally, the data collection device 102, the user device 134, the remote servers 145, the additional data sources 150, and/or the authorized entities 160 can use the data to train and otherwise improve the machine learning techniques and algorithms. In embodiments, the data collection device 102 and/or the remote servers 145 can depersonalize data for use without compromising sensitive information of patients, such as the identity of a patient. For example, the data collection device 102 and/or the remote servers 145 may depersonalize data when the data is used to train the machine learning techniques and algorithms to protect individuals' sensitive information while allowing the machine learning techniques and algorithms to be improved. In another example, the data collection device 102 and/or the remote servers 145 depersonalizes data of other individuals when data associated with multiple individuals is used to gain insights about a specific individual, such as the patient P. The data collection device 102, the user device 134, the additional data sources 150, and/or the authorized entities 160 may receive depersonalized data for the analysis and processing.

The data collection device 102, the user device 134, the remote servers 145, the additional data sources 150, and/or the authorized entities 160 may use data to perform real time alerting without the need for specialist review before the alert is sent. For example, t data collection device 102, the user device 134, the remote servers 145, the additional data sources 150, and/or the authorized entities 160 may analyze the data as the data is collected to determine if the data indicates that the patient P has any health concerns, whether immediately life threatening or a condition that should be evaluated in the future. The data collection device 102, the user device 134, the remote servers 145, the additional data sources 150, and/or the authorized entities 160 may send, display, or otherwise effectuate an alert to a specialist for review to determine if the flagged health concern is present in the patient P. For example, the data collection device 102 may display an alert graphic on the display 112 and generate an alert sound via the speaker system 108. The data collection device 102 may continuously monitor health data of the patient P, so the data collection device 102 may generate an alert in real time if the medical data changes to indicate a health concern.

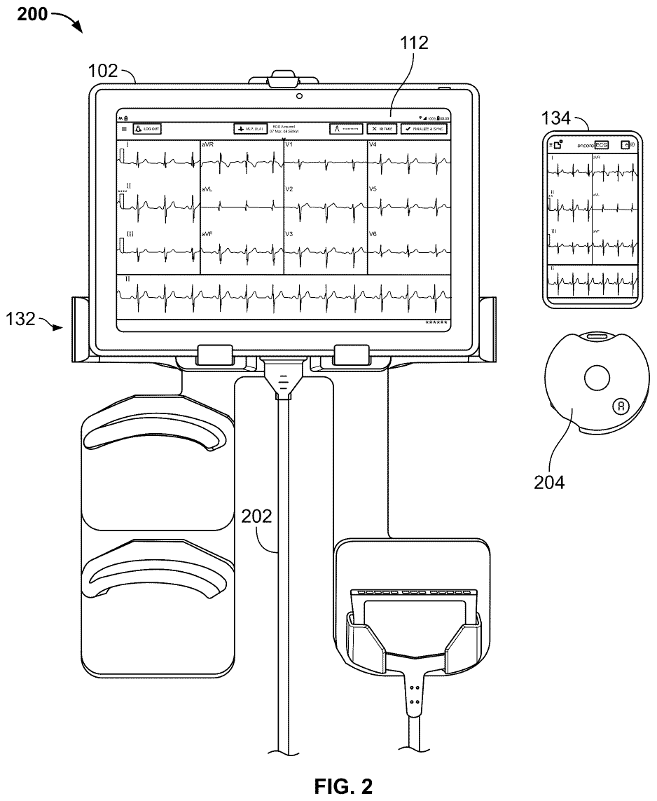

FIG. 2 is an illustration 200 of an example data collection device 102. The illustration includes the data collection device 102, the docking station 132, the user device 134, and a connection 202. In the illustration, the data collection device 102 is a portable tablet device that can be removed from the docking station 132 for transportation. The connection 202 may be part of the docking station 132 and may be the ECG connection system 130 or the ECG connection system 130 may be part of the connection 202. The connection 202 may also provide power to the data collection device 102 and provide connections to other sensors and data collection devices (e.g., a connection to the telemetry system 118. As shown in FIG. 2, the display 112 is displaying medical data (e.g., ECG data of the patient P). The user device 134 is also displaying the medical data.

The data collection device 102 can also include a standalone acquisition system 204. The acquisition system 204 can connect to the connection 202 and/or other sensor connections for collecting patient data. For example, the acquisition system 204 can connect to any number of leads to collect ECG data of the patient P. The acquisition system 204 can communicate with the data collection device 102 wirelessly and/or via a wired connection to provide the sensor data to the data collection device 102, and the acquisition system 204 can provide the data in real-time so the data collection device 102 can display the data in real-time and perform other real-time operations.

The acquisition system 204 can be a light-weight, portable system so a patient can move around during device collection. Additionally, the acquisition system 204 can include a battery or other power source to enable continuous collection of data for longer periods of time. For example, the acquisition system 204 can continuously collect data for twenty-four hours, forty-eight hours, seventy-two fours, etc. in various examples. Therefore, the acquisition system 204 may be implemented in a hospital, clinic, ambulance, or remote location (e.g., a home), providing improved mobility and flexibility to the user acquiring the ECG data from a patient. The acquisition system 204 may include an internal controller and execute a stored program fixed on physical non-transient medium to collect data and otherwise operate according to the program and according to the readings of the sensors, which may sense the connection of the ECG lead wires, the connection of a charging cord, and/or the quality of the transferred data. The controller may also communicate with user interface elements of the acquisition system 204, such as including a display screen and an actuator or input to start and stop data collection.

The acquisition system 204 may have a round housing made of a lightweight durable material, such as, for example, a plastic, polycarbonate, or acrylonitrile-butadiene-styrene. The housing may also be made of a durable, non-plastic material. The housing can be sized to fit to be portable (e.g., sized to be placed within a standard pocket) so that the module may be conveniently carried by a user before, during, and after the acquisition system 204 acquires data. The housing 70 may be approximately two to five inches in diameter by and a few inches wide. The width of the housing may be dependent on the size of the internal power source and the desired operation time on a single charge. The housing may include a slot to attach the housing to a lanyard or clip.

FIGS. 3-11 illustrate various interfaces the display 112 can display for data analysis and processing. FIG. 3 is a login interface 300 for logging in to the data collection device 102. The display 112 may display the login interface 300 when there is not a current user of the data collection device 102. The data collection device 102 may not perform operations for a user until the user logs in via the login interface 300. The login interface 300 includes an RFID element 302 and a manual login element 304. A user may tap an RFID device at the position indicated by the RFID element 302, and the RFID system 124 can receive an RF signal. The user identification system 122 can use the RF signal to identify the user and log the user into the data collection device 102. Alternatively, the user can manually input a username and password in the manual login element 304 or input a biometric characteristic. The biometric system 123 may receive the biometric characteristic, and the user identification system 122 can use the biometric characteristic to identify the user and log the user into the data collection device 102.

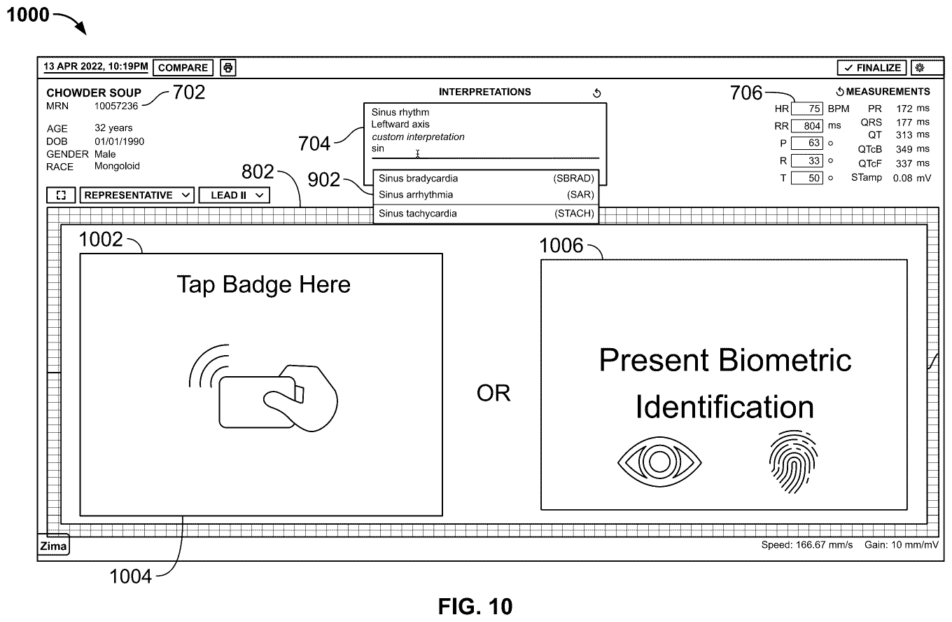

In some embodiments, the data collection device 102 may require user authentication when saving data, modifying data, inputting interpretations, and/or the like. The data collection device 102 may display a prompt for user authentication, such as the RFID element 302 or similar element to prompt a user to authenticate with a RFID device, a prompt to authenticate using the biometric system 123, and/or the like. FIG. 10 illustrates an example is described in more detail herein.

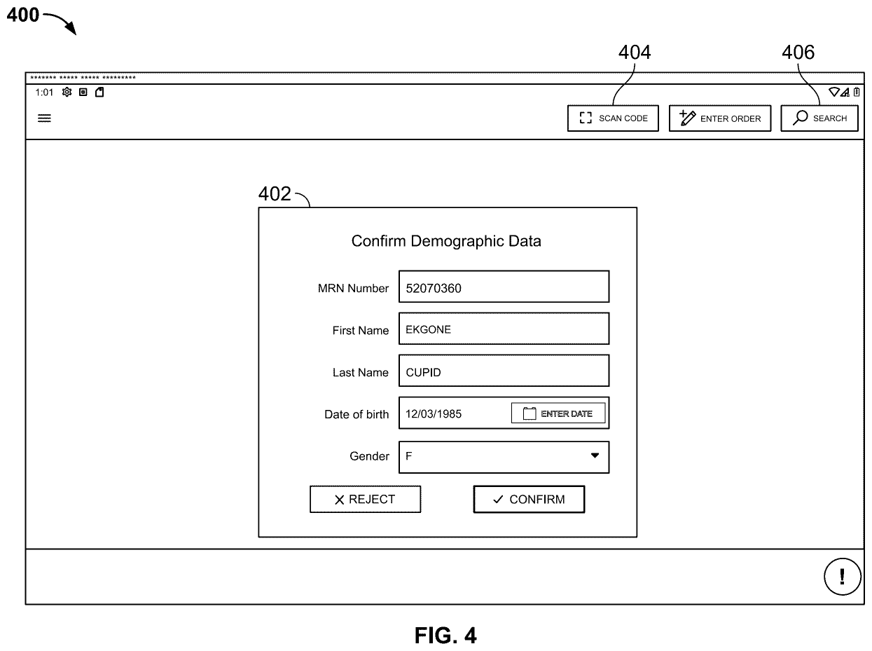

FIG. 4 is a patient data interface 400 for identifying patient information. The patient data interface 400 includes a patient information element 402, a scan code input 404, and a patient search input 406. The patient information element 402 may include patient information such as a patient number, a patient first name, a patient last name, a patient date of birth, a patient gender, and/or the like, A data collection device 102 user can input, review, reject, and confirm patient information using the patient information element 402. The user can select the scan code input 404 to cause the image capture device 106 to capture an image of a machine-readable code. The data collection device 102 may determine patient information using the machine-readable code and populate the patient information in the patient information element 402 for the user to review, adjust, reject, and confirm the patient information. The user can select the patient search input 406 to search for patient information, such as patient information stored by the storage system 128, the remote servers 145, or some other system. Once, the user selects patient information, the data collection device 102 can populate the patient information in the patient information element 402 for the user to review, adjust, reject, and confirm the patient information.

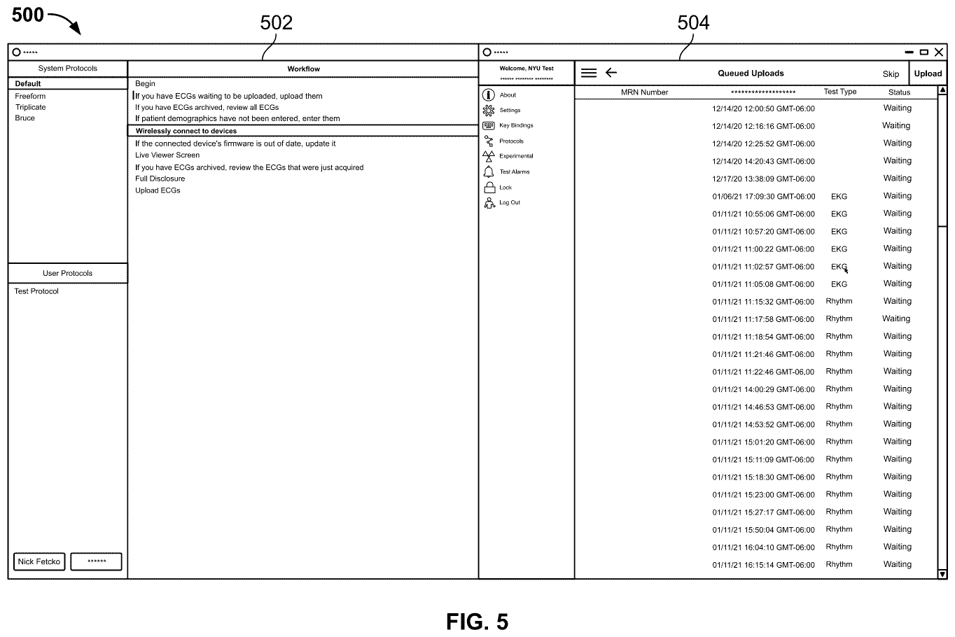

FIG. 5 is a workflow interface 500 for managing a workflow and uploading data. The data collection device 102 may provide a workflow for a user logged into the data collection device 102. The workflow may include tasks to be completed, such as selecting patient information, collecting patient data, reviewing data, approving data, sending data to other systems such as the remote servers 145, maintenance of the data collection device 102. The workflow interface 500 includes a workflow element 502 and an upload element 504. The workflow element 502 may populate with tasks based on the identity of the user logged into the data collection device 102, because different users may be assigned different tasks. The user may review the workflow element 502 to perform tasks, add and remove tasks, reorganize tasks, and/or the like. The upload element 504 may a list of data and the upload status of the data. The user may upload data, add or remove data to be uploaded, and/or the like using the upload element 504.

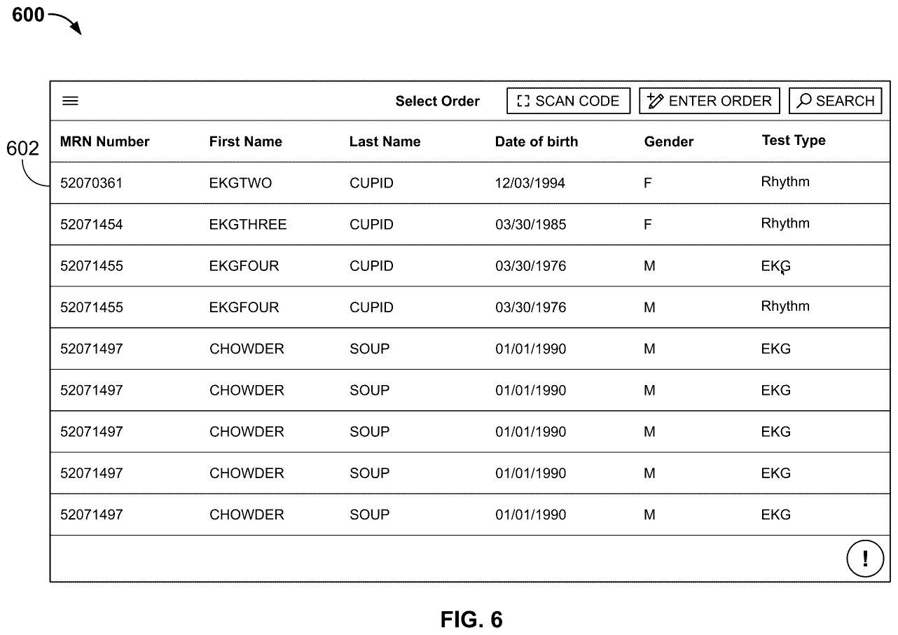

FIG. 6 is a patient queue interface 600 for managing patient data collection. The user logged into the data collection device 102 may have a list of patients to collect data for, based on the workflow as shown in FIG. 5 for example. The patient queue interface 600 includes a patient queue element 602. The user may review the patient list, add or remove patient data collection elements from the patient queue element 602, update the patient queue element 602 as data collection is performed, and/or the like.

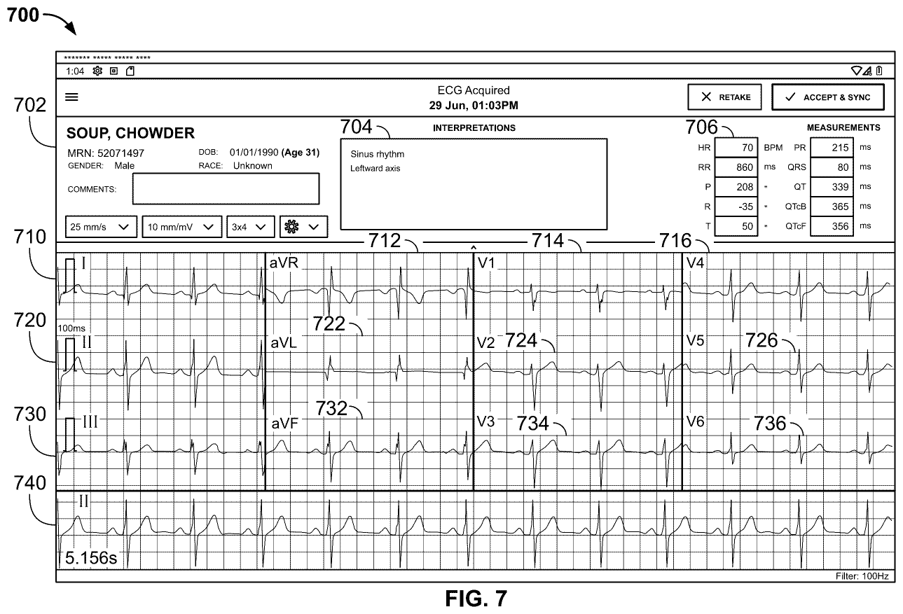

FIG. 7 is a multi-lead medical data interface 700 for viewing and analyzing patient data. The multi-lead medical data interface 700 allows a user to view, analyze, and process patient data. The multi-lead medical data interface 700 includes a patient information element 702 that includes the information of the patient associated with the displayed data. The multi-lead medical data interface 700 also includes an interpretations element 704, and the data collection device 102, another system, and/or the user may input analysis of the patient data in the interpretations element 704. The multi-lead medical data interface 700 also includes a measurements element 706. The measurements element 706 may include patient measurements, such as heart rate, RR interval, PR interval, P axis, R axis, T axis, QRS, QT, QTcB, QTcF, and/or the like.

The multi-lead medical data interface 700 also includes a first lead element 710, an aVR element 712, a V1 element 714, a V4 element 716, a second lead element 720, an aVL element 722, a V2 element 724, a V5 element 726, a third lead element 730, an aVF element 732, a V4 element 734, a V6 element 736, and an extended second lead element 740. These elements may be waveforms of data collected by the respective leads of the ECG connection system 130. The data collection device 102, another system, and/or the user may analyze and process this data.

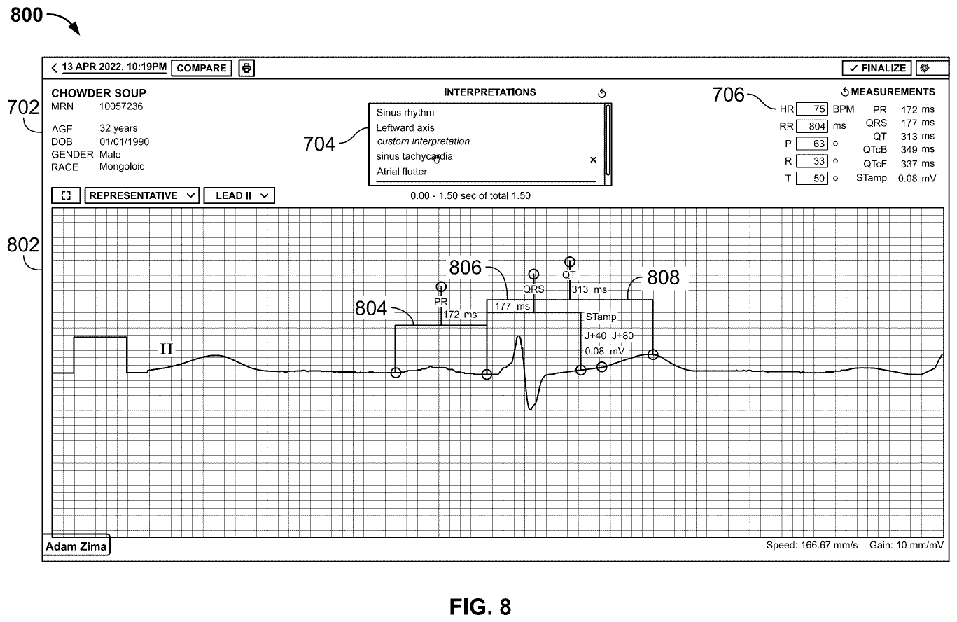

FIG. 8 is a single-lead medical data interface 800 for viewing and analyzing patient data. The single-lead medical data interface 800 includes the patient information element 702, the interpretations element 704, the measurements element 706, and a single-lead element 802. The single-lead element 802 may be a waveform of data from a lead of the ECG connection system 130. The single-lead element 802 includes a PR estimation 804, a QRS estimation 806, and a QT estimation 808. The data collection device 102 and/or another system may estimate the PR estimation 804, the QRS estimation 806, and the QT estimation 808. The user may adjust the PR estimation 804, the QRS estimation 806, and the QT estimation 808 such as via the touch I/O system 114. The display 112 may display additional analysis and processing of data in other examples.

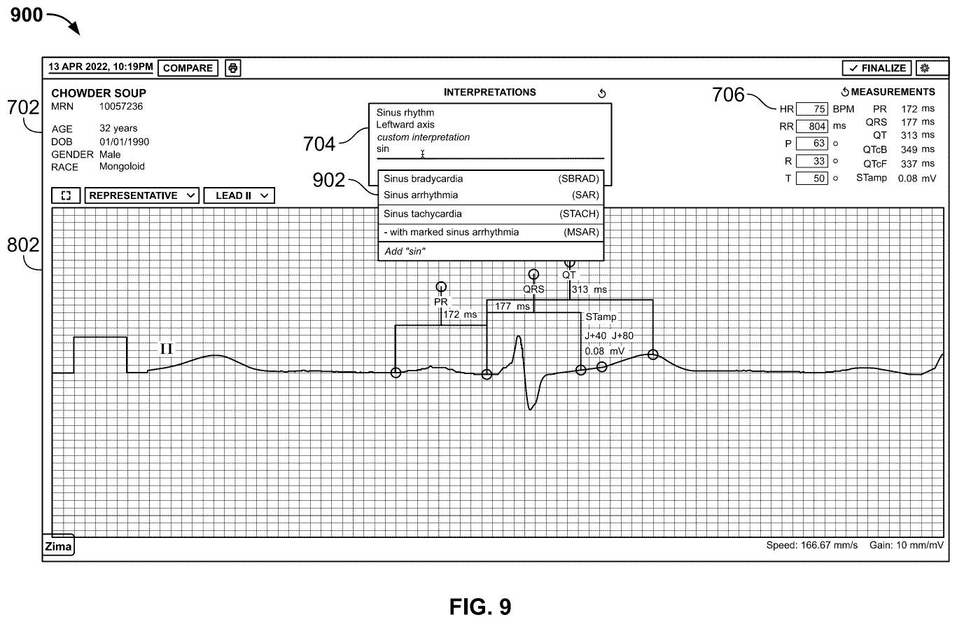

FIG. 9 is the single-lead medical data interface 800 for inputting interpretations of patient data. The single-lead medical data interface 800 includes an interpretations input element 902 in FIG. 9. The interpretations input element 902 may include a list of selectable interpretations of the data, and the user may select interpretations to be included in the interpretations element 704. The list of selectable interpretations may be listed based on suggested interpretations, such as interpretations the data collection device 102 and/or other systems may determine to be applicable based on analyzing and processing the data.

FIG. 10 is an authentication interface 1000 for authenticating a user. The authentication interface 1000 may include an authentication element 1002 displayed to instruct a user to perform authentication. In this embodiment, the authentication element 1002 includes an RFID prompt 1004 and a biometric prompt 1006. However, the authentication element 1002 may instruct a user to perform authentication with one or more prompts in other examples (e.g., just the RFID prompt 1004, just the biometric prompt 1006, a user credential prompt for manual input, a different combination of prompts, etc.). The RFID prompt 1004 instructs the user to present a RFID device for the RFID system 124. The biometric prompt 1006 instructs the user to present a biometric identifier for the biometric system 123.

The authentication element 1002 may be displayed on different interfaces, such as the interfaces shown in FIGS. 4-9, in response to a user action or other event that requires user authentication. For example, the authentication element 1002 may be displayed in response to collection of ECG data that needs a doctor to approve as valid before storage, in response to a user inputting interpretations, and/or the like.

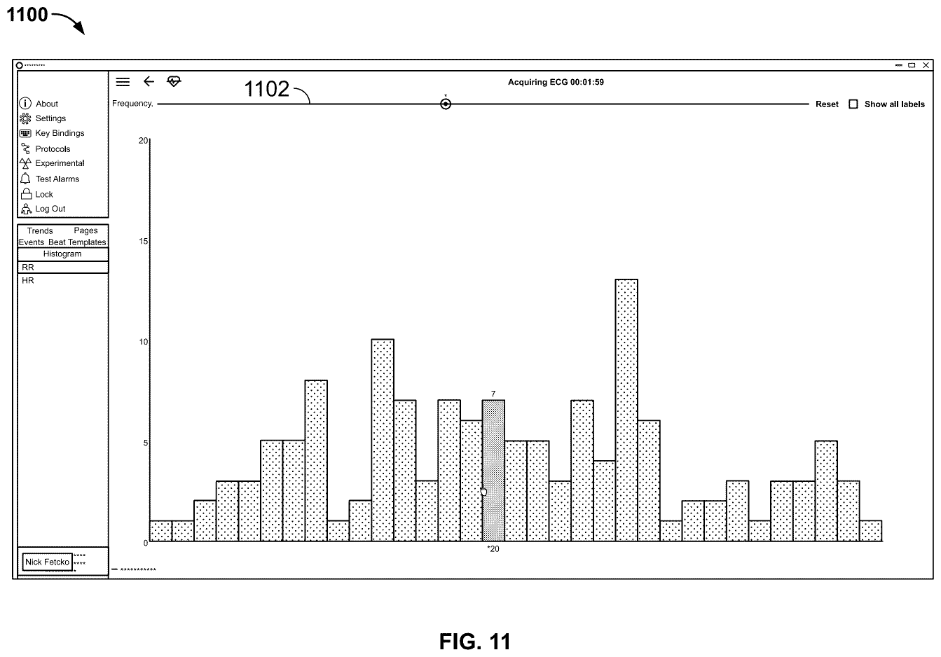

FIG. 11 is an interface 1100 for viewing and evaluating patient data. The interface 1100 includes a histogram 1102. The histogram 1102 illustrates the patient P data compared to other patient data. For example, the histogram 1102 may be the RR interval of the patient P compared to other patient RR intervals. The data collection device 102 may display the patient P data in different formats and compared in different ways to provide different options for data analysis.

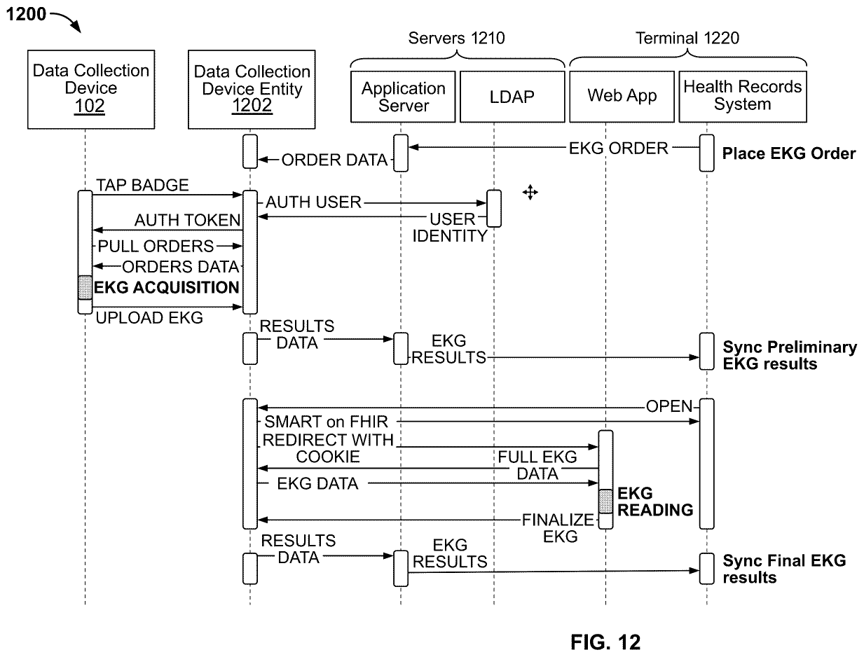

FIG. 12 is a block diagram of a communications flow 1200 between systems for data collection, storage, analysis, and processing. The communications flow 1200 can include the data collection device 102 or entity 1202 associated with the data collection device 102 receiving an order for health data such as EKG data. The order can originate from an entity such as a health records system (e.g., terminal 1220). The data collection device 102 can be used to collect data for the order. For example, the data collection device 102 can display the login interface 300 for a user to login, collect data, and the like.

The communications flow 1200 can include the data collection device 102 and/or the entity associated with the data collection device 102 to send preliminary results (e.g., results not verified by a medical professional). The data can be verified, and the data collection device 102 and/or the entity 1202 associated with the data collection device 102 can send medical data such as the final results (e.g., results verified by a medical professional). Servers 1210 can manage authorizing users, authenticating or otherwise verifying the data, and sending received data to a terminal 1220. The terminal 1220 can integrate and/or communicate with a centralized repository for adding the data to the centralized repository, providing data to devices, analyzing the data, and/or the like.

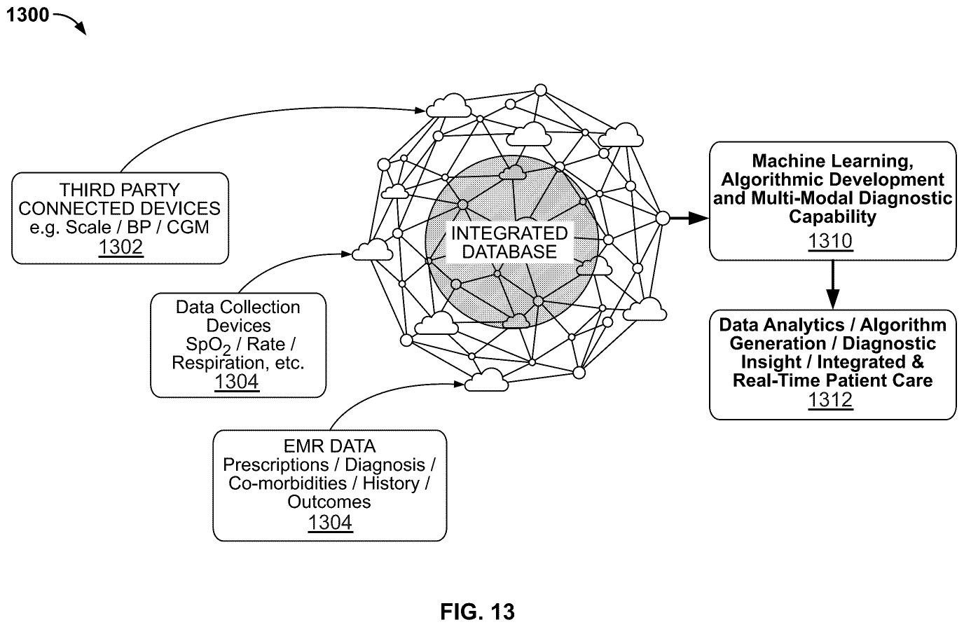

FIG. 13 is a block diagram of a centralized repository 1300 for data storage, analysis, and processing. The centralized repository 1300 can include data from various sources, including third party devices 1302, data collection devices 1302 (e.g., from the data collection device 102), EMR data 1304, and the like. Machine learning techniques 1310, including machine learning models, artificial intelligence, algorithms, and/or the like, can be used to analyze data and generate insights, such as for determining health concerns and otherwise diagnosing patients. Thus, the centralized repository 1300 can be utilized for patient diagnostic capabilities, to enable remote access to data, and so on. Data analytics processes 1312 can be performed on the data and outputs of machine learning techniques 1310 to train the machine learning techniques 1310, evaluate diagnostic capabilities of the machine learning techniques 1310, predict health concerns, and provide integrated and real-time patient care.

The example embodiments described herein may be implemented using hardware, software, or a combination thereof and may be implemented in one or more computer systems or other processing systems. However, the manipulations performed by these example embodiments were often referred to in terms, such as entering, which are commonly associated with mental operations performed by a human operator. No such capability of a human operator is necessary, in any of the operations described herein. Rather, the operations may be completely implemented with machine operations. Useful machines for performing the operation of the example embodiments presented herein include general purpose digital computers or similar devices.

From a hardware standpoint, a CPU typically includes one or more components, such as one or more microprocessors, for performing the arithmetic and/or logical operations required for program execution, and storage media, such as one or more memory cards (e.g., flash memory) for program and data storage, and a random-access memory, for temporary data and program instruction storage. From a software standpoint, a CPU typically includes software resident on a storage media (e.g., a memory card), which, when executed, directs the CPU in performing transmission and reception functions. The CPU software may run on an operating system stored on the storage media, such as, for example, UNIX or Windows, iOS, Linux, and the like, and can adhere to various protocols such as the Ethernet, ATM, TCP/IP protocols and/or other connection or connectionless protocols. As is well known in the art, CPUs can run different operating systems, and can contain different types of software, each type devoted to a different function, such as handling and managing data/information from a particular source or transforming data/information from one format into another format. It should thus be clear that the embodiments described herein are not to be construed as being limited for use with any particular type of server computer, and that any other suitable type of device for facilitating the exchange and storage of information may be employed instead.

A CPU may be a single CPU, or may include plural separate CPUs, wherein each is dedicated to a separate application, such as, for example, a data application, a voice application, and a video application. Software embodiments of the example embodiments presented herein may be provided as a computer program product, or software, which may include an article of manufacture on a machine accessible or non-transitory computer-readable medium (i.e., also referred to as “machine readable medium”) having instructions. The instructions on the machine accessible or machine-readable medium may be used to program a computer system or other electronic device. The machine-readable medium may include, but is not limited to, optical disks, CD-ROMs, and magneto-optical disks or other type of media/machine-, readable medium suitable for storing or transmitting electronic instructions. The techniques described herein are not limited to any particular software configuration. They may find applicability in any computing or processing environment. The terms “machine accessible medium”, “machine readable medium” and “computer-readable medium” used herein shall include any non-transitory medium that is capable of storing, encoding, or transmitting a sequence of instructions for execution by the machine (e.g., a CPU or other type of processing device) and that cause the machine to perform any one of the methods described herein. Furthermore, it is common in the art to speak of software, in one form or another (e.g., program, procedure, process, application, module, unit, logic, and so on) as taking an action or causing a result. Such expressions are merely a shorthand way of stating that the execution of the software by a processing system causes the processor to perform an action to produce a result.

The various examples and teachings described above are provided by way of illustration only and should not be construed to limit the scope of the present disclosure. Those skilled in the art will readily recognize various modifications and changes that may be made without following the examples and applications illustrated and described herein, and without departing from the true spirit and scope of the present disclosure.

What is claimed is:

1. A data collection device, comprising:
an electrocardiogram (“ECG”) system;
an ECG connection system operable to connect to a patient;
a display; and
a controller operable to:
cause the ECG system to collect patient data via the ECG connection system, wherein the patient data is sampled;
cause the display to display the patient data; and
access patient data of a plurality of other patients stored in a centralized repository; and
evaluate the patient data to determine a patient condition, including comparing the patient data to the patient data of the plurality of other patients.

2. The data collection device of claim 1, further comprising an image capture device, wherein the controller is operable to:
cause the image capture device to capture an image of a machine-readable code; and
identify the patient using the machine-readable code.

3. The data collection device of claim 1, further comprising a microphone system, wherein the controller is operable to:
cause the microphone system to receive a verbal command; and
adjust the operation of the data collection device based on the verbal command.

4. The data collection device of claim 1, further comprising a user identification system, wherein the controller is operable to cause the user identification system to identify a user of the data collection device.

5. The data collection device of claim 4, wherein the user identification system comprises a biometric system, wherein to identify the user comprises to:
cause the biometric system to receive a biometric characteristic; and
identify the user based on the biometric characteristic.

6. The data collection device of claim 4, wherein the user identification system comprises a Radio Frequency Identification (RFID) system, wherein to identify the user comprises to:
cause the RFID system to receive a RF signal; and
identify the user based on the RF signal.

7. The data collection device of claim 4, wherein the controller is operable to:
cause the display to display a user login page, wherein to identify the user is in response to displaying the user login page.

8. The data collection device of claim 4, wherein the controller is operable to:
cause the display to display a user verification element; and
receive verification of the patient data via the user verification element.

9. The data collection device of claim 1, further comprising a telemetry system, wherein the controller is operable to:
cause the telemetry system to collect patient telemetry data of the patient.

10. The data collection device of claim 1, wherein the controller is further operable to:
cause the data collection device to the data to remote servers.

11. A method comprising:
receiving data from a plurality of sources, wherein:
the data comprises a plurality of formats, and
the plurality of sources each comprise a data collection device, wherein the data collection device collects clinical grade data;
storing the data in a centralized repository; and
analyzing the data to generate insights.

12. The method of claim 11, further comprising:
receiving, from a specialist, a selection of a modality for using the data;
determining a portion of the data relevant to the modality; and
causing a system associated with the specialist to access the data.

13. The method of claim 11, further comprising processing the data to prepare the data for analysis.

14. The method of claim 11, wherein analyzing the data to generate the insights comprises:
determining a health concern; and
causing a system associated with one of the plurality of sources to receive an alert of the health concern.

15. The method of claim 11, wherein the data comprises (i) ECG data, (ii), Holter and event monitoring data, (iii) vital sign data, (iv) telemetry monitoring data, (v) cardiac stress testing data, or (vi) any combination of (i), (ii), (iii), (iv), and (v).

16. The method of claim 11, wherein the data is associated with a patient, further comprising:
receiving a request to access a medical record;
creating the medical record using the data; and
causing a system associated with the patient to access the data.

17. The method of claim 11, further comprising:
receiving, from an individual associated with the data, authorization for an entity to access the data;
receiving, from the entity, a request to access the data; and
causing a system associated with the entity to access the data.

18. The method of claim 11, wherein analyzing the data to generate the insights comprises:
using a machine learning technique to analyze the data; and
generating one of (i) a preventative care recommendation, (ii) a diagnostic care recommendation, (iii) a treatment recommendation, or (iv) any combination of (i), (ii), and (iii).

19. A data collection device, comprising:
an ECG system;
an ECG connection system comprising a plurality of leads operable to connect to a patient;
a display; and
a controller operable to:
cause the ECG system to collect patient data via the ECG connection system, wherein the patient data is sampled;
cause the display to display the patient data for the plurality of leads on a same view;
access patient data of a plurality of other patients stored in a centralized repository; and
evaluate the patient data to determine a patient condition, including comparing the patient data to the patient data of the plurality of other patients.

20. The data collection device of claim 19, wherein the controller is operable to:
cause the display to display a user verification element; and
receive verification of the patient data via the user verification element.
